(19) United States
(12) Patent Application Publication
(10) Pub. No.: US 2020/0149013 A1
(43) Pub. Date: May 14, 2020

(54) Method of Selecting Viruses for the Treatment of Cancer

(71) Applicant: Chris Coleclough, Germantown, TN (US)

(72) Inventor: Chris Coleclough, Germantown, TN (US)

(21) Appl. No.: 16/188,388

(22) Filed: Nov. 13, 2018

(51) Int. Cl. C12N 7/00; A61K 35/768

(52) U.S. Cl. CPC C12N 7/00; A61K 35/768

(57) ABSTRACT

The present disclosure presents a method of genetically selecting an RNA virus to treat cancer. The method involves choosing an RNA virus, exposing it to the cancer cells, followed by exposure to non-target cells to select for the RNA virus that does not bind non-target cells, followed by repeating the process until a treatment RNA virus is obtained.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC

Not Applicable

### DESCRIPTION

### Field of the Invention

A specific RNA virus can be genetically directed to treat cancer through a method of repeated virus selection for growth on cancer cells, and for non-binding of non-target cells. RNA viruses, specifically enteroviruses and alphaviruses, lack the genetic repair and correction mechanisms of entities containing double-stranded DNA genomes. RNA viruses are therefore among the most variable human pathogens, and can potentially be adapted/selected to treat cancer. The present disclosure seeks to exploit this variability to genetically direct the chosen RNA virus through the disclosed method by selecting RNA viruses which, during the disclosed method, specifically target human tumors, for example, carcinoma of the pancreas, of the breast, or of the colon, while not adhering to or attacking non-target cells.

### BACKGROUND OF THE INVENTION

Some research has been conducted using an RNA virus to treat cancerous cells. However, this research did not use the presently disclosed directed evolutionary technique to select the RNA virus to specifically treat the cancer. See, Taylor et al., Viruses as an Aid to Cancer Therapy: Regression of Solid and Ascites Tumors in Rodents After Treatment with Bovine Enterovirus, Proc. Nat. Acad. Sci. USA, Vol. 68, No. 4, pp. 836-840, April 1971; and Quetglas, et al., Alphavirus Vectors for Cancer Therapy, Virus Res. 2010 November; 153(2):179-96. doi: 10.1016/j.virusres.2010.07.027. Epub 2010 Aug. 6. In more recent studies with oncolytic viruses, often molecular genetics is used rather than directed positive and negative selection strategies. Furthermore, because most adults have developed antibodies against RNA viruses (because these viruses are frequently encountered during childhood), the RNA viruses disclosed in other studies may be neutralized prior to being able to treat the cancer.

### SUMMARY OF THE INVENTION

The present disclosure reveals a method of genetically directing RNA viruses through a selection process to produce a treatment for cancer. The method involves choosing an RNA virus, exposing virus to the cancer cells to select viruses able to kill cancer cells, collecting the RNA virus, exposing the RNA virus to non-target cells, collecting the RNA virus that does not bind the non-target cells, and using resultant viruses to repeat the selection process once or more than once.

During the method, the RNA virus may also be exposed to anti-viral factors including but not limited to interferon alpha, interferon beta, interferon gamma, interleukin 6, T cells, and/or normal human sera to select viruses that are resistant to anti-viral factors.

During any step in the method, the RNA virus in the process of selection can be amplified in an appropriate environment to ensure sufficient quantities of the selected RNA virus for the method to continue.

### BRIEF DESCRIPTION OF THE SEVERAL VIEWS OF THE DRAWING

FIG. 1 Is a one-line drawing of the disclosed method.

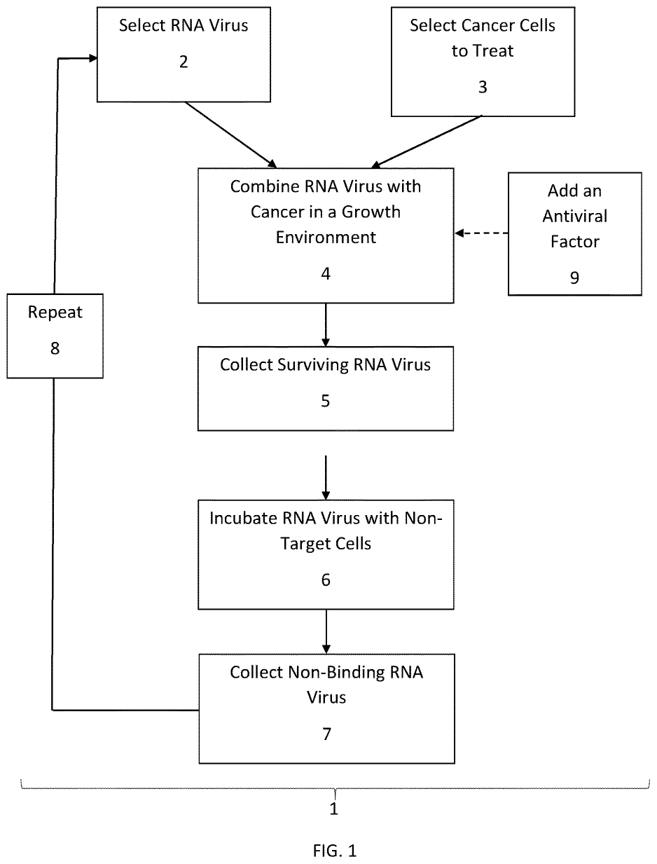

### DETAILED DESCRIPTION OF THE INVENTION

The present disclosure reveals a method 1 of genetically directing an RNA virus for the treatment of cancer through a selection process, wherein said method comprises choosing at least one RNA virus 2 for the treatment of cancer. The RNA virus is chosen from enteroviruses and/or alpha viruses. Cancer cells 3 are selected from at least one of, but are not limited to, pancreatic cancer, breast cancer, and colon cancer.

Once a specific RNA virus has been chosen, it is combined with the cancer cells in a growth environment and allowed to grow until cancer cell lysis 4 (disintegration of a cell by Rupture of the cell wall or membrane).

Once some or all cancer cells are lysed, the surviving RNA viruses are collected 5. The RNA virus is then incubated 6 with non-target cells. The intent of this incubation process is to select RNA viruses that are incapable of binding the non-target cells. After incubation 6, the non-binding RNA viruses are collected 7.

The RNA viruses collected 7 from the incubation 6 are then used to repeat 8 the method of positive and negative selection a plurality of times, to include once or more than once.

During any of the plurality of repetitions of the method, anti-viral factors 9 may be added to assure resistance of the selected virus. The anti-viral factor is chosen from at least one of interferon alpha, interferon beta, interferon gamma, interleukin 6, T cells, and/or normal human sera.

During any step in the method, the RNA virus in the process of selection can be amplified in an appropriate environment to ensure sufficient quantities of the selected RNA virus for the method to continue.

What is claimed:

1. A method of genetically directing an RNA virus for the treatment of cancer comprising:
choosing at least one RNA virus from enteroviruses and/or alphaviruses to propagate for the treatment of cancer;
combining the chosen RNA virus in a growth medium with the cancer cells and incubation until some or all cancer cells are lysed;
collect surviving RNA viruses;
incubate the RNA virus with non-target cells;
collect RNA viruses that do not bind non-target cells; and
repeat the method of selection a plurality of times.

2. The method of claim 1 wherein, during at least one repetition of the method an anti-viral factor selected from at least one of interferon alpha, interferon beta, interferon gamma, interleukin 6, T cells, and/or normal human serum is added to the growth medium.
